(19) United States
(12) Patent Application Publication
(10) Pub. No.: US 2025/0180569 A1
(43) Pub. Date: Jun. 5, 2025

(54) SURFACED MODIFICATION OF NALNF4 NANOPARTICLES WITH BISPHOSPHONATE DERIVATIVES FOR MASS CYTOMETRY APPLICATIONS

(71) Applicants: Standard Bio Tools Canada Inc., Markham (CA); The Governing Council of the University of Toronto, Toronto (CA)

(72) Inventors: Mahtab Abtahi, Toronto (CA); Daniel Majonis, Markham (CA); Ladan Gheiratmand, Markham (CA); Hyungjun Cho, Toronto (CA); Loryn P. Arnett, Toronto (CA); Mitchell A. Winnik, Toronto (CA)

(21) Appl. No.: 18/842,367

(22) PCT Filed: Mar. 1, 2023
(86) PCT No.: PCT/US2023/014273

§ 371 Date: Aug. 28, 2024

Related U.S. Application Data
(60) Provisional application No. 63/352,615, filed on Jun. 15, 2022.
(60) Provisional application No. 63/315,524, filed on Mar. 1, 2022.

(51) Int. Cl. G01N 33/58; B82Y 15/00; C09K 11/02; C09K 11/77; G01N 15/10; G01N 15/1433

(52) U.S. Cl. CPC G01N 33/587; C09K 11/025; C09K 11/7773; G01N 15/1433; B82Y 15/00; G01N 2015/1006

(57) ABSTRACT

Described herein are nanoparticles comprising NaLnF4, wherein Ln includes all non-radioactive lanthanide elements, or NaYF4, and methods of making and using the same. The nanoparticles may optionally be modified with PEG or zwitterionic polymers containing a bisphosphonate end group, wherein the bisphosphonate end group optionally comprises an aminohexyl group. As described herein. NaLnF4 or NaYF4 nanoparticles may be used as high-sensitivity reagents for mass cytometry.

### CROSS-REFERENCES TO RELATED APPLICATIONS

The present application claims priority to U.S. Provisional Pat. Appl. No. 63/352,615, filed on Jun. 15, 2022, and to U.S. Provisional Pat. Appl. No. 63/315,524, filed on Mar. 1, 2022, the entire contents of both of which are incorporated herein by reference in their entirety for all purposes.

### BACKGROUND

Mass cytometry (MC) is a high throughput multiparameter bioanalytical technique for single cell analysis. Imaging mass cytometry (IMC) is an extension of MC that couples a laser ablation system to the mass cytometer enabling one to measure cellular markers on tissue sections. Reagents for MC and IMC™ include Ab conjugated metal-chelating polymers carrying ˜150 metal ions. To achieve higher sensitivity, reagents with a larger number of metal atoms per mass tag are desirable.

### BRIEF SUMMARY

Mass cytometry (MC) is a high throughput multiparameter bioanalytical technique for single cell analysis. In this technique, cell suspensions are labeled with antibodies (Abs) tagged with heavy metal isotopes. Cells are injected individually but stochastically into an inductively coupled plasma time-of-flight mass spectrometer, where the signal at the detector is directly proportional to the number of ions per Ab-conjugate. MC is described in PCT Application No. PCT/US2021/049667, the contents of which are incorporated herein by reference for all purposes. MC instruments cover the mass range between 89 (Y) to 220 (Bi) and can analyze up to 100 parameters per cell. Imaging mass cytometry (IMC) is an extension of MC that couples a laser ablation system to the mass cytometer enabling one to measure cellular markers on tissue sections. Reagents for MC and IMC™ include Ab conjugated metal-chelating polymers carrying ˜150 metal ions. To achieve higher sensitivity, reagents with a larger number of metal atoms per mass tag are desirable. As described herein, lanthanide nanoparticles (LnNPs) in the form of NaLnF₄ are good candidates for this purpose, as a 10-20 nm diameter LnNP contains ˜10⁵ Ln ions. In some examples, NaLnF₄ nanoparticles (NPs) as described herein may be used in biological applications such as in vitro cell labelling, in vivo imaging, and cell tracking.

As described herein, NaLnF₄ or NaYF₄ NPs may be used as high-sensitivity reagents for mass cytometry. These NPs may be uniform in size, colloidally stable in phosphate buffers (i.e., PBS) and biological media, and carry functionality for antibody attachment and avoid non-specific interactions with cells or tissue samples. Preventing non-specific binding has been a significant challenge. An approach to minimize or reduce non-specific binding is to modify the NP surface with polyethylene glycol (PEG). However, an alternative to PEG is surface modification with zwitterionic polymers. Zwitterionic moieties include positively and negatively charged groups linked by a short carbon chain. Without being limited by theory, zwitterionic polymers may have decreased biological interactions relative to PEG, which may be attributable to the super-hydrophilic nature of zwitterions.

Provided herein is a nanoparticle, the nanoparticle including NaLnF₄, wherein Ln includes all non-radioactive lanthanide elements, or NaYF₄. Optionally, the nanoparticle described herein is modified with PEG or zwitterionic polymers containing a bisphosphonate end group, wherein the bisphosphonate end group optionally includes an aminohexyl group.

Provided also herein is a method of analyzing a cell, the method including introducing the cell to the nanoparticle as described herein.

Also provided herein is a mass cytometry system, the mass cytometry system including any one of the nanoparticles as described herein.

Also provided herein is a mixture of nanoparticles, the mixture including a plurality of nanoparticles, each nanoparticle being a nanoparticle of any one of the nanoparticles as described herein.

Without wishing to be bound by any particular theory, there can be discussion herein of beliefs or understandings of underlying principles relating to embodiments of the present invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 shows a diagram of a nanoparticle core with a corona according to embodiments of the present invention.

FIG. 2A shows that Oleate coated nanoparticles were transferred to aqueous media using citrate exchange according to embodiments of the present invention.

FIG. 2B shows a narrow size distribution of citrate-coated nanoparticles according to embodiments of the present invention.

FIG. 2C shows a transmission electron microscopy (TEM) image of the citrate-coated nanoparticles according to embodiments of the present invention.

FIG. 3 shows synthesis of PEG-Neridronate and ligand exchange according to embodiments of the present invention.

FIG. 4A shows a graph of the intensity versus diameter for NPs in water according to embodiments of the present invention.

FIG. 4B shows a graph of the intensity versus diameter for mPEG₂₀₀₀-Ner coated NPs in PBS according to embodiments of the present invention.

FIG. 4C shows a TEM image of mPEG₂₀₀₀-Ner coated NPs according to embodiments of the present invention.

FIG. 5A shows the signal in the ⁸⁹Y channel of the sample in 3% BSA prepared in Example 4 according to embodiments of the present invention.

FIG. 5B shows the signal in the ¹⁷⁴Yb channel of the sample in 3% BSA prepared in Example 4 according to embodiments of the present invention.

FIG. 6A shows synthesis of zwitterionic polymer PSBMA-Ner according to embodiments of the present invention.

FIG. 6B shows three different nanoparticles tested: NPs incubated with 100% PSBMA-Ner, NPs incubated with 50% mPEG-Ner and 50% PSBMA-Ner, and NPs incubated with 100% mPEG-Ner according to embodiments of the present invention.

FIG. 7A shows characterization of NPs with 50% mPEG-Ner and 50% PSBMA-Ner according to embodiments of the present invention.

FIG. 7B shows characterization of NPs with 100% PSBMA-Ner according to embodiments of the present invention.

FIGS. 8A and 8B show non-specific binding results of the NPs according to embodiments of the present invention.

FIG. 9 is a table showing surface polymer compositions of NPs according to embodiments of the present invention.

FIG. 10 shows NPs tested for different lengths according to embodiments of the present invention. The NPs tested include NPs with 100% mPEG₂₀₀₀-Ner, NPs with about 50% mPEG₂₀₀₀-Ner and about 50% PSBMA-Ner, NPs with about 50% N₃-PEG₂₀₀₀-Ner and about 50% PSBMA-Ner, NPs with about 50% mPEG₅₀₀₀-Ner and about 50% PSBMA-Ner, and NPs with about 50% N₃-PEG₅₀₀₀-Ner and about 50% PSBMA-Ner. N₃ is an azide functional group.

FIG. 11 shows the non-specific binding results from suspension mass cytometry experiments of the NPs according to embodiments of the present invention.

FIG. 12 shows using DBCO-azide click chemistry to conjugate antibodies (CD45RO or PD-1) onto NPs with about 50% N₃-PEG₅₀₀₀-Ner and about 50% PSBMA-Ner according to embodiments of the present invention.

### DETAILED DESCRIPTION

Mass cytometry (MC) is a high throughput multiparameter bioanalytical technique for single cell analysis. In this technique, cell suspensions are labeled with antibodies (Abs) tagged with heavy metal isotopes. Cells are injected individually but stochastically into an inductively coupled plasma time-of-flight mass spectrometer, where the signal at the detector is directly proportional to the number of ions per Ab-conjugate. MC is described in PCT Application No. PCT/US2021/049667, the entire contents of which are incorporated herein by reference for all purposes. MC instruments cover the mass range between 89 (Y) to 220 (Bi) and can analyze up to 100 parameters per cell. Imaging mass cytometry (IMC) is an extension of MC that couples a laser ablation system to the mass cytometer enabling one to measure cellular markers on tissue sections. Reagents for MC and IMC™ include Ab conjugated metal-chelating polymers carrying ˜150 metal ions. To achieve higher sensitivity, reagents with a larger number of metal atoms per mass tag are desirable. Lanthanide nanoparticles (LnNPs) in the form of NaLnF₄ as described herein are good candidates for this purpose, as a 10-20 nm diameter LnNP contains ˜10⁵ Ln ions. In some examples. NaLnF₄ nanoparticles (NPs) as described herein may be used in biological applications such as in vitro cell labelling, in vivo imaging, and cell tracking.

NaLnF₄ or NaYF₄ NPs may be used as high-sensitivity reagents for mass cytometry. These NPs may be uniform in size, colloidally stable in phosphate buffers (i.e., PBS) and biological media, and carry functionality for antibody attachment and avoid non-specific interactions with cells or tissue samples. Preventing non-specific binding has been a significant challenge. An approach to minimize or reduce non-specific binding is to modify the NP surface with polyethylene glycol (PEG). However, an alternative to PEG is surface modification with zwitterionic polymers. Zwitterionic moieties include positively and negatively charged groups linked by a short carbon chain. Without being limited by theory, zwitterionic polymers may have decreased biological interactions relative to PEG, which may be attributable to the super-hydrophilic nature of zwitterions.

Some embodiments described herein use uniform NaYF₄:Yb/Er NPs (approximately 25 nm diameter) modified with PEG or zwitterionic polymers containing a bisphosphonate end-group.

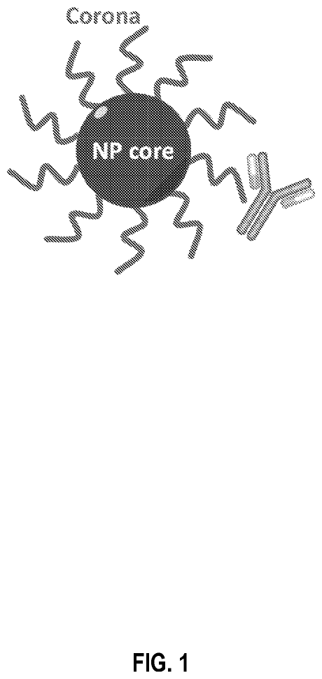

As described herein and as shown in FIG. 1, a nanoparticle core may include a corona, where the corona may be attached to an antibody. In some embodiments, the core has a narrow size distribution (e.g., coefficient of variation [CV]<5%). Without being limited by theory, a narrow size distribution of the core aids in keeping the number of ions per mass tag consistent. Without being limited by theory, the corona promotes colloidal stability in a phosphate buffer. In some examples, the corona may suppress non-specific binding with cells. In some examples, the corona also may have functionalities for antibody conjugation. The diameter of the nanoparticle core as described herein may be from about 10 nm to about 50 nm, which may be measured from a transmission electron microscopy (TEM) image. The overall hydrodynamic diameter (dₕ) of the nanoparticle in solution including the corona can be measured, for example, by Dynamic Light Scattering.

Neridronate (Ner) is an aminohexyl bisphosphonate. As described herein, in some examples, PEG is conjugated with Ner (PEG-Ner) in a 1:1 ratio using the Ner amine functionality. In other examples, as an alternative to PEG, a poly(sulfobetaine methacrylate) (PSBMA), a zwitterionic polymer, was prepared by RAFT polymerization, and Ner was conjugated to one end of the PSBMA (PSBMA-Ner). In some examples, nanoparticles (NPs) comprising a coating comprising PEG-Ner, PSBMA-Ner, and/or PEG-Ner/PSBMA-Ner mixtures via ligand exchange are also described herein. In some examples, the modified NPs were characterized by investigating their morphology, colloidal stability, surface polymer composition, and non-specific binding with tissue samples. In certain non-limiting examples, no change in morphology was observed after ligand exchange with either PEG-Ner or PSBMA-Ner. In some examples, NPs coated with PEG-Ner, PSBMA-Ner and/or PEG-Ner/PSBMA-Ner showed long-term colloidal stability in PBS (e.g., colloidally stable for at least 24 hours, 7 days, 28 days, or 64 days). In some embodiments, IMC™ tests showed PEG-Ner coated NPs as described herein displayed significant amounts of non-specific binding, however, PSBMA-Ner modified NPs as described herein had significantly reduced non-specific binding, even when used in a mixture with PEG-Ner. In some examples as described herein, functional-PEGs can introduce Abs for targeted binding studies in conjugation with the PSBMA to decrease non-specific binding.

### 1. Nanoparticles Comprising NaLnF₄

Described herein is a nanoparticle including MLnF₄ or MYF₄, wherein M is Li, Na, K, Rb, or Cs, and wherein Ln includes all non-radioactive lanthanide elements. Described also herein is a nanoparticle including NaLnF₄, wherein Ln includes all non-radioactive lanthanide elements, or NaYF₄. In some embodiments, Ln includes La, Ce, Pr, Nd, Sm, Eu, Gd, Tb, Dy, Ho, Er, Tm, Yb, Lu, or a combination thereof.

In some embodiments, the nanoparticle further includes a dopant. In some embodiments, the dopant includes Yb or Er.

In some embodiments, the nanoparticle is modified with PEG or zwitterionic polymers containing a bisphosphonate end group.

In some embodiments, the nanoparticle is modified with a zwitterionic polymer containing a bisphosphonate end group, wherein the bisphosphonate end group includes an aminohexyl bisphosphonate. In some embodiments, the aminohexyl bisphosphonate comprises Neridronate. In some embodiments, the aminohexyl bisphosphonate comprises alendronate. In some embodiments, the alendronate is conjugated to poly(sulfobetaine methacrylate) (PSBMA) at one end. In some embodiments, the alendronate is conjugated to PEG at one end.

In some embodiments, the nanoparticle is modified with PEG or zwitterionic polymers containing a bisphosphonate end group, and the nanoparticle is modified with poly(sulfobetaine methacrylate) (PSBMA). In some embodiments the nanoparticle is modified with the zwitterionic polymer, and the bisphosphonate end group of the zwitterionic polymer comprises an aminohexyl bisphosphonate, and wherein the nanoparticle is modified with poly(sulfobetaine methacrylate) (PSBMA).

In some embodiments the nanoparticle is modified with the zwitterionic polymer, and the bisphosphonate end group of the zwitterionic polymer comprises an aminohexyl bisphosphonate that comprises Neridronate, and wherein the nanoparticle is modified with poly(sulfobetaine methacrylate) (PSBMA). In some embodiments, the Neridronate is conjugated to one end, wherein the Neridronate is conjugated to the PSBMA (PSBMA-Ner). In some embodiments, the nanoparticle is further modified with PEG. In some embodiments, the PEG is PEG₂₀₀₀ or PEG₅₀₀₀. In some embodiments, an azide functional group is conjugated to the PEG. In other embodiments, the PEG is conjugated to Neridronate (PEG-Ner).

In some embodiments, the nanoparticle as described herein is modified with PSBMA conjugated to Neridronate at one end (PSBMA-Ner) and with PEG conjugated to Neridronate at one end (PEG-Ner). In some embodiments, the PSMBA-Ner is 30% to 40%, 40% to 50%, or 50% to 60% of the total PSBMA-Ner and PEG-Ner modifications.

In some embodiments the nanoparticle is modified with the zwitterionic polymer, and the bisphosphonate end group of the zwitterionic polymer comprises an aminohexyl bisphosphonate that comprises alendronate, and wherein the nanoparticle is modified with poly(sulfobetaine methacrylate) (PSBMA). In some embodiments, the alendronate is conjugated to one end, wherein the alendronate is conjugated to the PSBMA (PSBMA-alendronate). In some embodiments, the nanoparticle is further modified with PEG. In some embodiments, the PEG is PEG₂₀₀₀ or PEG₅₀₀₀. In some embodiments, an azide functional group is conjugated to the PEG. In other embodiments, the PEG is conjugated to alendronate (PEG-alendronate).

In some embodiments, the nanoparticle as described herein is modified with PSBMA conjugated to alendronate at one end and with PEG conjugated to alendronate at one end (PEG-alendronate). In some embodiments, the PSMBA-alendronate is 30% to 40%, 40% to 50%, or 50% to 60% of the total PSBMA-alendronate and PEG-alendronate modifications.

In some embodiments, the nanoparticle as described herein may have a diameter in a range from 10 nm to 50 nm (e.g., from 15 nm to 45 nm, from 20 nm to 40 nm, or from 25 nm to 35 nm). In some examples, the diameter is measured by transmission electron microscopy.

In some embodiments, the nanoparticle as described herein may comprise at least 10⁵ Ln ions (e.g., at least 10³, 10⁴, 10⁵, 10⁶, 10⁷, or 10⁸ Ln ions).

The nanoparticle as described herein can be attached to one or more antibodies or one or more other bioaffinity agents. In some embodiments, the nanoparticle comprises an antibody. In some embodiments, the nanoparticle comprises another bioaffinity agent. In some embodiments, the nanoparticle comprises at least one or more antibodies and at least one or more other bioaffinity agents. In some embodiments, the antibody includes, but is not limited to, CD45RO or PD-1. In some embodiments, the antibody includes an IgG or IgM from a mouse, rabbit, rat, goat, hamster, or similar species. In some embodiments, the antibody includes an antibody suitable for use in flow cytometry, immunohistochemistry, or immunofluorescence. In some embodiments, the other bioaffinity agent includes, but is not limited to, oligonucleotides, avidin-type proteins, aptamers, lectins, or other biomolecules that specifically bind a target biomolecule.

### 2. Methods of Analyzing Cells

Also described herein is a method of analyzing a cell, the method comprising introducing to the cell any one of the nanoparticles as described herein. In some examples, the method further includes analyzing a mixture of cells and one or more of any one of the nanoparticles described herein. In some examples, the analyzing step includes analyzing the mixture of cells and the nanoparticles by mass cytometry. In some examples, the method includes delivering the mixture of cells and the nanoparticles to a mass cytometry system, where the mass cytometry system includes an inductively coupled plasma source and an analyzer. In some examples, the method further includes an ignition step, where the mixture of cells and the nanoparticles is ignited by the inductively coupled plasma source. In some examples, the method further includes detecting the mixture of cells and the nanoparticles with the analyzer.

### 3. Mass Cytometry Systems

Also described herein is a mass cytometry system including any one of the nanoparticles as described herein. In some examples, the mass cytometry system comprises one or more of the nanoparticles as described herein. The mass cytometry system may include an inductively coupled plasma source and an analyzer. The analyzer may include a mass spectrometer or an imaging mass spectrometer.

### 4. Mixtures of Nanoparticles

Also described herein is a mixture of nanoparticles comprising a plurality of nanoparticles, each nanoparticle being any one of the nanoparticles as described herein. In some embodiments, the plurality of nanoparticles comprises at least 10, 100, 1,000, 10,000, 100,000, 1,000,000, or 10,000,000 nanoparticles.

The invention may be further understood by the following non-limiting examples.

### EXAMPLES

PSBMA was synthesized, and neridronate was attached to the end. Samples of NaYF₄:Yb/Er NPs were coated with PEG-neridronate, with PSBMA-neridronate, and/or with mixtures of PEG-neridronate and PSBMA-neridronate. Low non-specific binding was desired, because neither PEG-neridronate or PSBMA-neridronate had a targeting agent on the polymer. In these experiments PEG-neridronate showed substantial non-specific binding, while PSBMA-neridronate showed very low levels of non-specific binding. NaYF₄:Yb/Er NPs coated with mixtures of PEG-neridronate and PSBMA-neridronate also showed very low levels of non-specific binding. Inclusion of PEG-neridronate in the mixture allows for the use of a heterobifunctional PEG-neridronate in these mixed coatings to attach antibodies or other bioaffinity agents to the NPs.

Analogous materials may include alendronate replacing neridronate. For example, any nanoparticle with neridronate may have alendronate replace the neridronate.

### Example 1

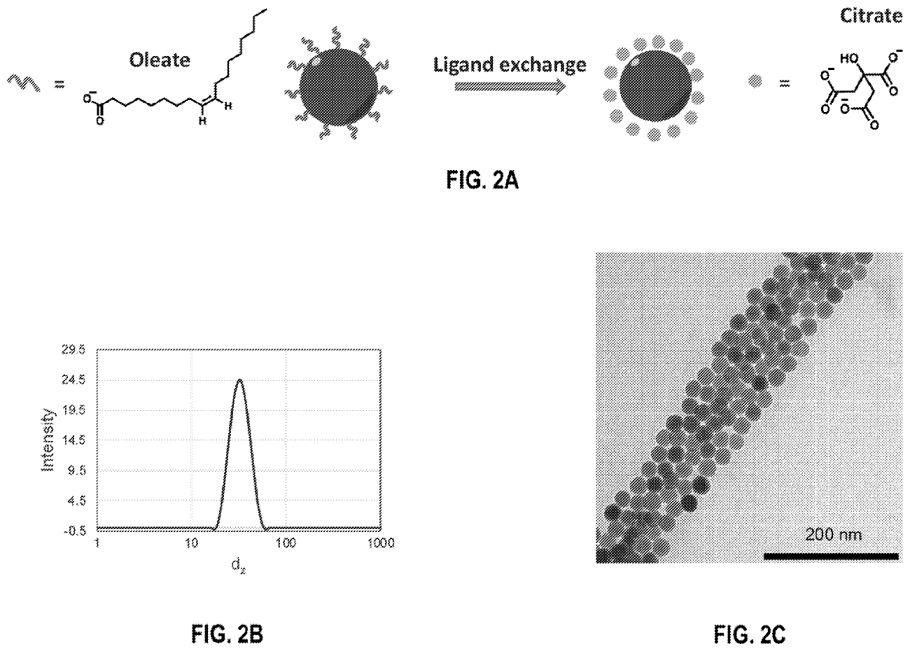

Oleate coated nanoparticles as described herein were transferred to aqueous media using citrate exchange, as shown in FIG. 2A. Without being limited by theory, citrate exchange may be fast, be safe, and allow long-term storage of nanoparticles.

As shown in FIG. 2B, a narrow size distribution of citrate-coated nanoparticles was observed. The z-average hydrodynamic diameter (d_{z}), which is expressed as an intensity-based harmonic mean, was 31.6 nm. The polydispersity index (PDI) was 0.03.

As shown in FIG. 2C, a transmission electron microscopy (TEM) image of the citrate-coated nanoparticles was obtained, showing that the mean diameter of nanoparticles (d_{TEM}) in TEM was 28 nm. No change in the morphology of NPs was observed after citrate exchange.

### Example 2

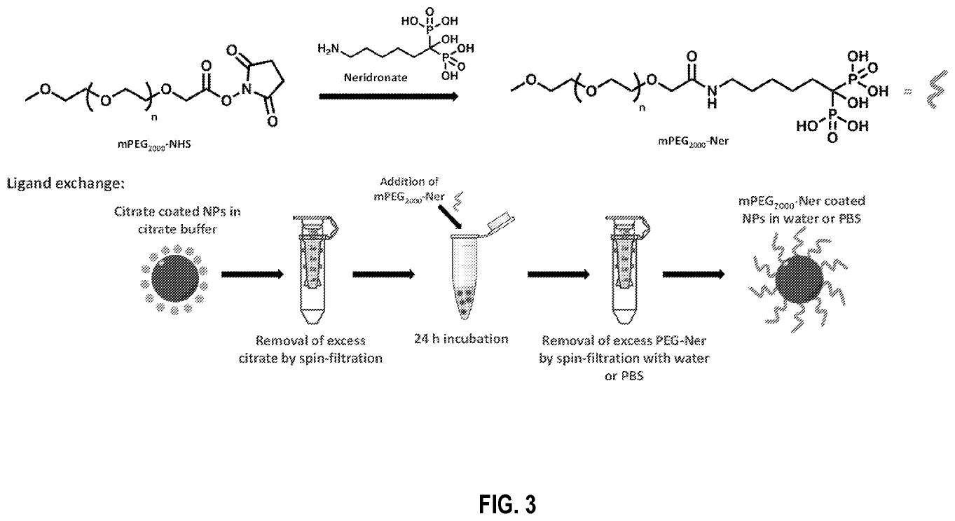

The synthesis of PEG-Neridronate and ligand exchange as described herein in one embodiment is shown in FIG. 3. As shown in FIG. 3, mPEG₂₀₀₀-NHS was combined with Neridronate to form mPEG₂₀₀₀-Ner. Nanoparticles as described herein coated with mPEG₂₀₀₀ were prepared by a ligand exchange procedure with citrate. Accordingly, a solution of nanoparticles as described herein coated with citrate and citrate buffer was prepared. Excess citrate in solution was removed by spin-filtration. Then, mPEG₂₀₀₀-NER was added to the solution, and the solution was allowed to incubate for twenty-four hours at room temperature. Excess mPEG₂₀₀₀-NER was removed by spin filtration with water, or optionally removed by spin filtration with PBS solution. Accordingly, mPEG₂₀₀₀-Ner coated nanoparticles as described herein were thus obtained in water or PBS.

### Example 3

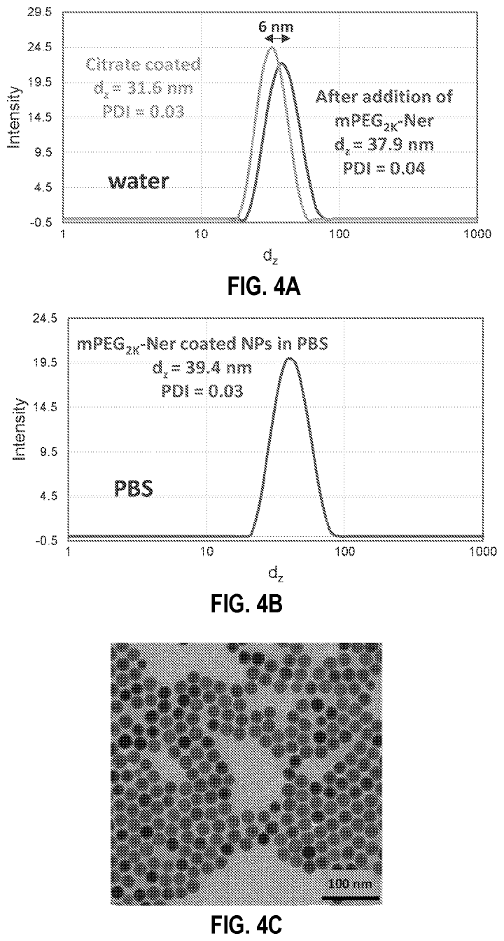

Some examples of nanoparticles as described herein were characterized with dynamic light scattering (DLS) and TEM. FIG. 4A shows a graph of the intensity versus diameter for the NPs in water. The citrate coated NPs shown in the graph of FIG. 2B are shown in the trace to the left in FIG. 4A. The mPEG₂₀₀₀-Ner coated NPs were observed to have an average diameter of 37.9 nm and a PDI of 0.04. The average diameter of the mPEG₂₀₀₀-Ner coated NPs relative to the citrate coated NPs was observed to increase by about 6.3 nm, as seen in FIG. 4A.

FIG. 4B shows a graph of the intensity versus diameter for mPEG₂₀₀₀-Ner coated NPs in PBS. The average diameter was observed to be 39.4 nm. The PDI was observed to be 0.03. In this example, the mPEG₂₀₀₀-Ner coated NPs were observed colloidally stable for at least 28 days in PBS.

FIG. 4C shows a TEM image of mPEG₂₀₀₀-Ner coated NPs. No change in morphology of NPs was observed to occur after coating with mPEG₂₀₀₀-Ner.

### Example 4

The non-specific binding of mPEG₂₀₀₀-Ner coated NaYF₄:Yb/Er NPs was tested with tissue samples prepared by an immunohistochemistry protocol. Normal human tonsil tissues underwent dewaxing, hydration, and antigen retrieval. The tissue sample then underwent blocking with BSA buffer. The tissue sample then was stained with a metal tagged antibody cocktail including antibodies and the NPs (without antibodies). The stained tissue sample was analyzed by Standard BioTools Inc. IMC™.

### Example 5

Because the antibodies were not on the NPs in Example 4, there should be no specific binding. Any signal in imaging mass cytometry is a result of non-specific binding.

The NP concentration on the tissue was 2×10¹⁰ NP/mL. Intensity less than 5 is background. Intensity greater than 5 is signal.

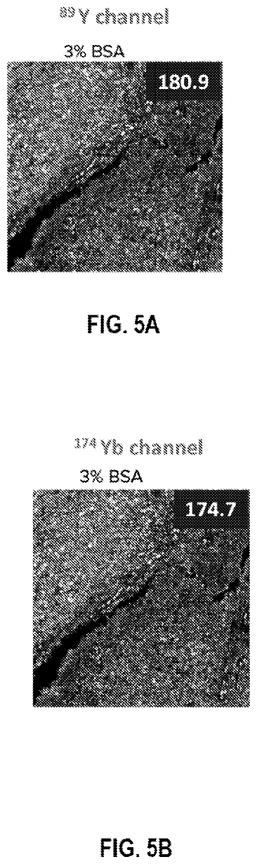

FIG. 5A shows the signal in the ⁸⁹Y channel of the sample in 3% BSA prepared in Example 4. The average intensity was 180.9. FIG. 5A shows non-specific binding. FIG. 5B shows the signal in the ¹⁷⁴Yb channel of the sample in 3% BSA prepared in Example 4. The average intensity was 174.7. FIG. 5B shows non-specific binding.

### Example 6

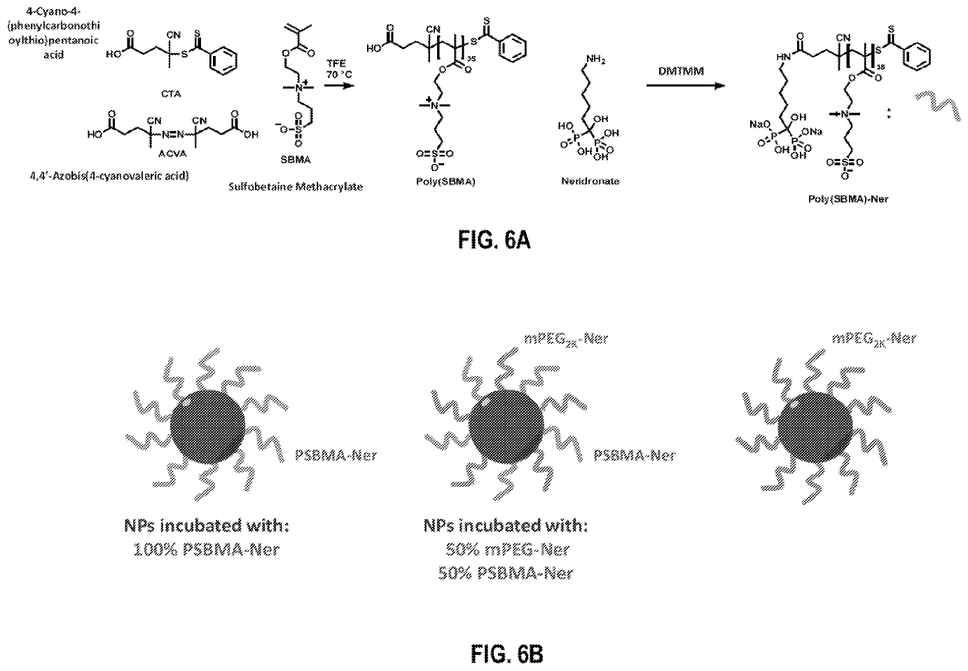

Zwitterionic polymers were added to attempt to reduce non-specific binding. FIG. 6A shows synthesis of zwitterionic polymer PSBMA-Ner.

FIG. 6B shows three different nanoparticles tested: NPs incubated with 100% PSBMA-Ner, NPs incubated with 50% mPEG-Ner and 50% PSBMA-Ner, and NPs incubated with 100% mPEG-Ner.

### Example 7

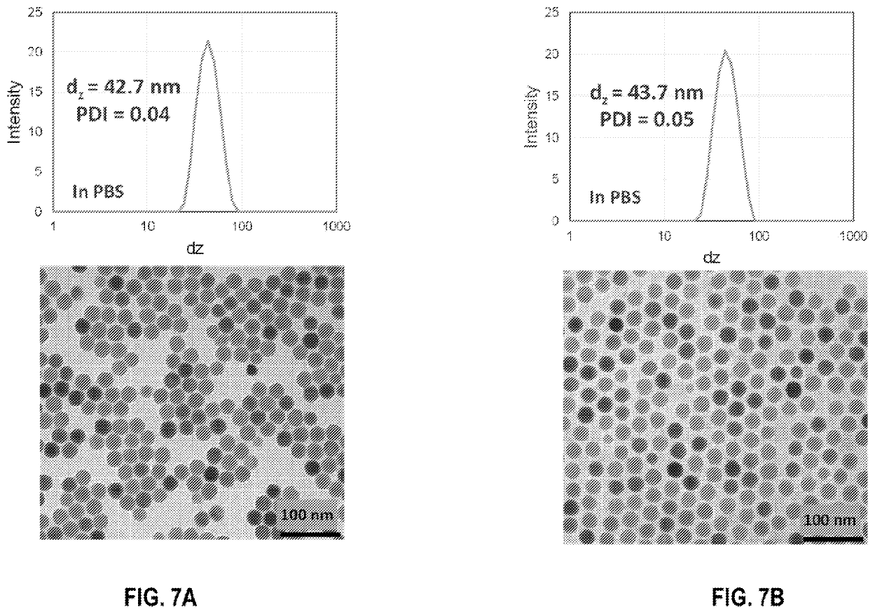

FIG. 7A shows characterization of NPs with 50% mPEG-Ner and 50% PSBMA-Ner. The average diameter for these NPs was observed 42.7 nm. The PDI was 0.04.

FIG. 7B shows characterization of NPs with 100% PSBMA-Ner. The average diameter was observed to be 43.7 nm. The PDI was 0.05.

Both samples were colloidally stable in PBS and have narrow size distributions by DLS. No change in morphology was observed by TEM.

### Example 8

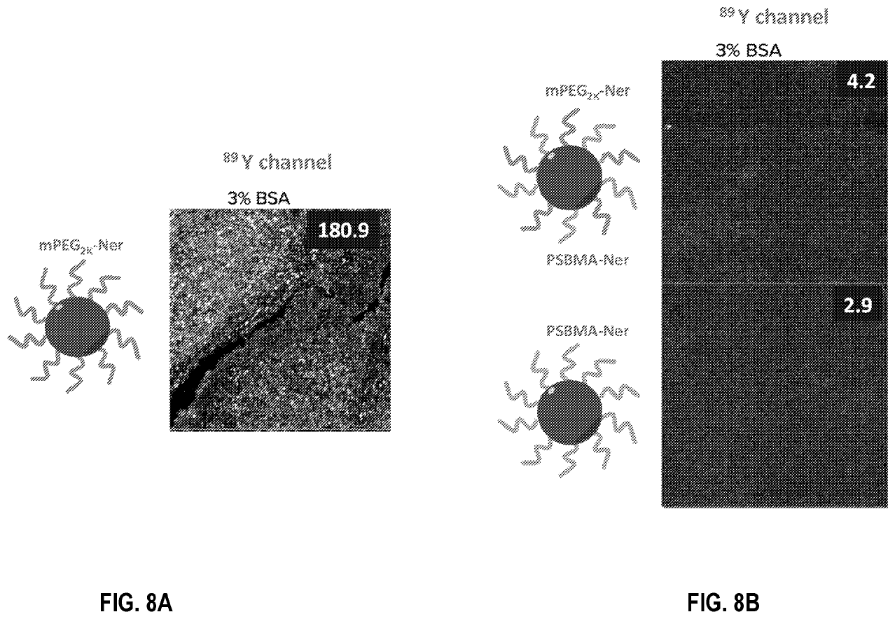

FIGS. 8A and 8B show non-specific binding results of the NPs described in FIG. 6B. FIG. 8A shows that NPs with 100% mPEG-Ner still show a signal (intensity of 180.9), indicating non-specific binding. FIG. 8B shows that NPs with 50% mPEG-Ner and 50% PSBMA-Ner and NPs with 100% PSBMA-Ner show a signal consistent with background (intensities of 4.2 for 50%/50% and 2.9 for 100% PSBMA-Ner). FIG. 8B indicates low non-specific binding when NPs are coated with PSBMA-Ner. The addition of zwitterionic polymer was observed to decrease the non-specific binding significantly, resulting in no signal observed.

### Example 9

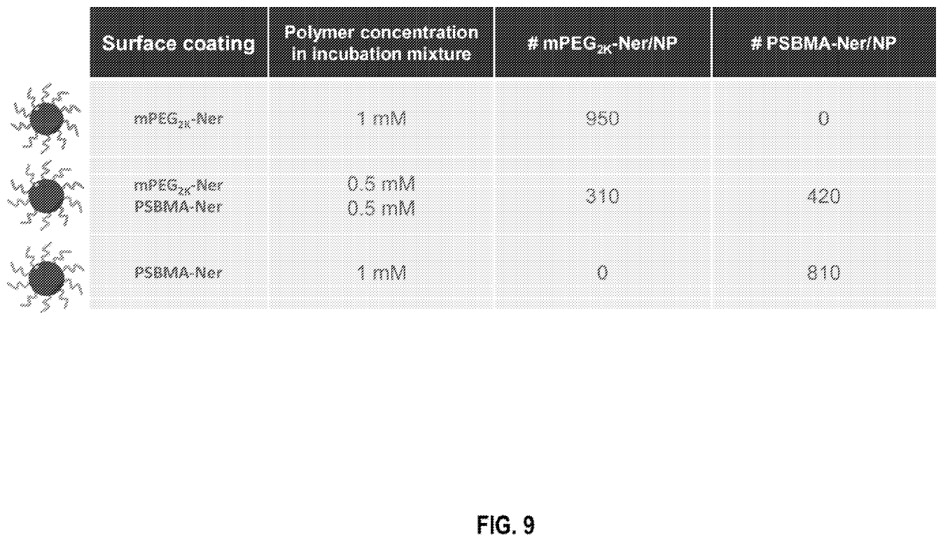

FIG. 9 is a table showing surface polymer compositions of NPs coated with polymers as described herein. NPs were incubated with (1) mPEG₂₀₀₀-NER (1 mmol/L (mM)): (2) mPEG₂₀₀₀-Ner (0.5 mM) and PSBMA-Ner (0.5 mM); or (3) PSBMA-Ner (1 mM). The surface composition of the NPs as described in FIG. 9 was quantified by ICP-OES (inductively coupled plasma-optical emission spectroscopy) measurements on ³¹P content from both polymers and ³²S content from PSBMA-Ner. The number of mPEG₂₀₀₀-Ner per NP and the number of PSBMA-Ner per NP were obtained for all three compositions, where the results are shown in FIG. 9.

### Example 10

PEGs with different lengths are studied for how they affect non-specific binding. PEGs with an average molecular weight of 2,000 and 5,000 were studied. A PEG with a higher molecular weight is longer than a PEG with a lower molecular weight.

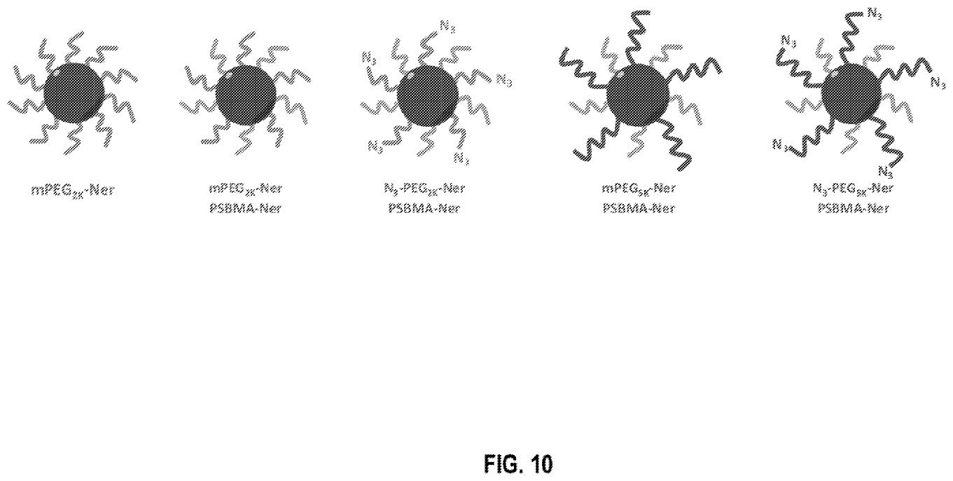

FIG. 10 shows NPs tested for different lengths. The NPs tested include NPs with 100% mPEG₂₀₀₀-Ner, NPs with about 50% mPEG₂₀₀₀-Ner and about 50% PSBMA-Ner, NPs with about 50% N₃-PEG₂₀₀₀-Ner and about 50% PSBMA-Ner, NPs with about 50% mPEG₅₀₀₀-Ner and about 50% PSBMA-Ner, and NPs with about 50% N₃-PEG₅₀₀₀-Ner and about 50% PSBMA-Ner. N₃ is an azide functional group.

### Example 11

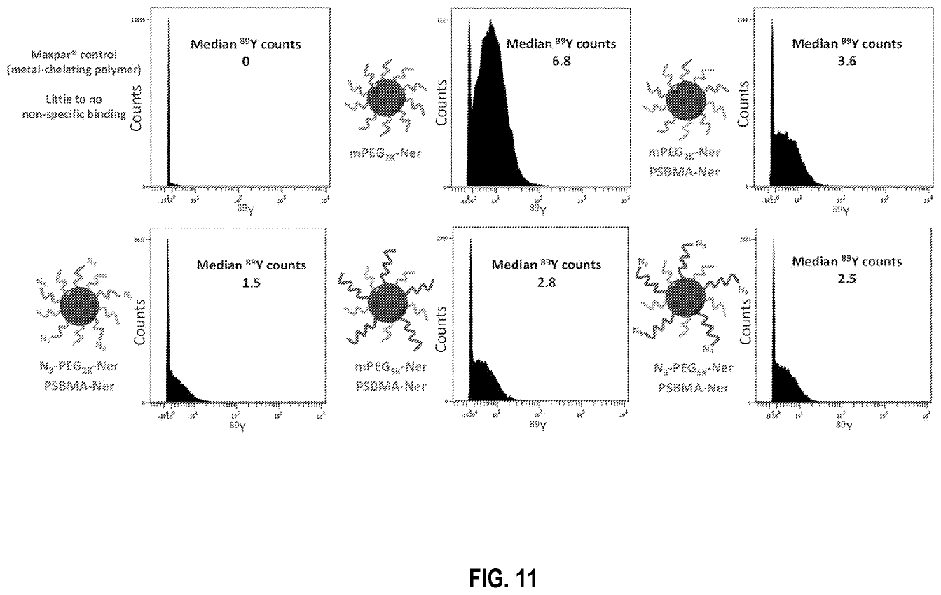

FIG. 11 shows the non-specific binding results from suspension mass cytometry experiments of the NPs as described in FIG. 10. The x-axis shows the signal intensity. The y-axis is the number of cells counted at each signal intensity. NPs with PEG₅₀₀₀ and PSBMA-Ner showed low non-specific binding similar to NPs with PEG₂₀₀₀ and PSBMA-Ner. A metal-chelating polymer (Maxpar®) is shown as a control with little to no non-specific binding, as shown in FIG. 11.

### Example 12

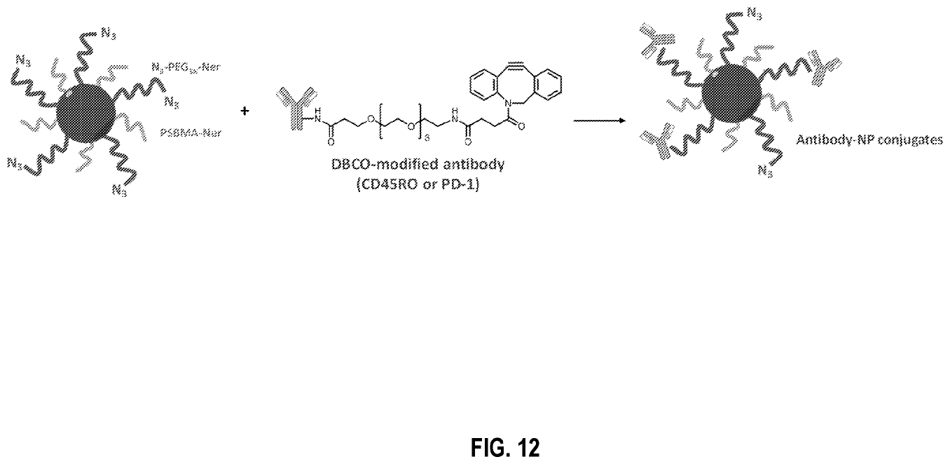

Antibodies were conjugated onto the NPs. FIG. 12 shows using DBCO-azide click chemistry to conjugate antibodies (CD45RO or PD-1) onto NPs with about 50% N₃-PEG₅₀₀₀-Ner and about 50% PSBMA-Ner.

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference. None is admitted to be prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list: for example “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure. It is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein. “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A nanoparticle, the nanoparticle comprising:
NaLnF₄, wherein Ln includes all non-radioactive lanthanide elements, or NaYF₄.

2. The nanoparticle of claim 1, further comprising a dopant.

3. The nanoparticle of claim 2, wherein the dopant comprises Yb and Er.

4. The nanoparticle of any one of claims 1 to 3, wherein the nanoparticle is modified with PEG or zwitterionic polymers containing a bisphosphonate end group.

5. The nanoparticle of claim 4, wherein the nanoparticle is modified with the zwitterionic polymer, and the bisphosphonate end group comprises an aminohexyl bisphosphonate.

6. The nanoparticle of claim 5, wherein the aminohexyl bisphosphonate comprises Neridronate.

7. The nanoparticle of claim 5, wherein the aminohexyl bisphosphonate comprises alendronate.

8. The nanoparticle of claim 7, wherein the alendronate is conjugated to poly(sulfobetaine methacrylate) (PSBMA) at one end.

9. The nanoparticle of claim 8, wherein the nanoparticle is further modified with alendronate conjugated to PEG at one end.

10. The nanoparticle of any of claims 4 to 6, wherein the nanoparticle is modified with poly(sulfobetaine methacrylate) (PSBMA).

11. The nanoparticle of claim 10, wherein the Neridronate is conjugated to one end (PSBMA-Ner).

12. The nanoparticle of claim 10 or 11, wherein the nanoparticle is further modified with PEG.

13. The nanoparticle of claim 12, wherein the PEG is PEG₂₀₀₀ or PEG₅₀₀₀.

14. The nanoparticle of claim 12 or 13, wherein an azide functional group is conjugated to the PEG.

15. The nanoparticle of any one of claims 12 to 13, wherein the PEG is conjugated to Neridronate (PEG-Ner).

16. The nanoparticle of claim 1, wherein the nanoparticle is modified with PSBMA conjugated to Neridronate at one end (PSBMA-Ner) and with PEG conjugated to Neridronate at one end (PEG-Ner).

17. The nanoparticle of claim 16, wherein the PSBMA-Ner is 30% to 40%, 40% to 50%, or 50% to 60% of the total PSBMA-Ner and PEG-Ner modifications.

18. The nanoparticle of any one of claims 1 to 17, wherein the diameter of the nanoparticle as measured by transmission electron microscopy is in a range from 10 nm to 50 nm.

19. The nanoparticle of any of claims 1 to 18, wherein the nanoparticle comprises at least 10⁵ Ln ions.

20. The nanoparticle of any one of claims 1 to 19, wherein the nanoparticle is attached to an antibody or other bioaffinity agent.

21. A method of analyzing a cell, the method comprising introducing the cell to the nanoparticle of any one of claims 1 to 20.

22. A mass cytometry system, the mass cytometry system comprising the nanoparticle of any one of claims 1 to 19.

23. A mixture of nanoparticles, the mixture comprising a plurality of nanoparticles, each nanoparticle being a nanoparticle of any one of claims 1 to 19.

24. The mixture of claim 23, wherein the plurality of nanoparticles comprises at least 10, 100, 1,000, 10,000, 100,000, 1,000,000, or 10,000,000 nanoparticles.
